(19) United States
(12) Patent Application Publication
(10) Pub. No.: US 2004/0023301 A1
(43) Pub. Date: Feb. 5, 2004

(54) Method for immobilizing lipid layers

(76) Inventor: Joachim Noller, Leipzig (DE)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/381,851

(22) PCT Filed: Sep. 25, 2001
(86) PCT No.: PCT/EP01/11036

(30) Foreign Application Priority Data
Sep. 26, 2000 (DE) 100 48 822.6

(51) Int. Cl. G01N 33/53; G01N 33/543

(52) U.S. Cl. 435/7.1; 436/518; 427/2.11

(57) ABSTRACT

A method is provided for immobilizing lipid layers on solid-body surfaces, in which method the solid-body surfaces are modified such that the properties, in particular with regard to diffusivity, of the lipid layers which are deposited on the surfaces to a large extent correspond to those of lipid layers which have not been immobilized. In the method, the solid-body surface is firstly modified with molecules such that an essentially hydrophilic surface area is formed. In a second procedural step, lipid layers, in particular double lipid layers, are deposited on this modified surface.

The invention relates to a method for preparing lipid layers which are immobilized on surfaces of pulverulent solid bodies, and to modified solid-body surfaces, immobilized lipid layers and a kit which is suitable for preparing them.

In the last few years, solid body-supported double lipid layers (termed bilayers in that which follows) and solid body-supported functional biomembranes in which membrane proteins are immobilized have gained in importance in the fields of drug screening, chromatography and biosensor technology and for lipid binding studies. The first solid body-supported bilayers which were described in the literature were only separated from the solid body surface by a water film of from 1 to 2 nm in thickness (Tamm, L. K.; McConnell, H. M. Biophysical Journal 1985, 47, 105-113; Johnson, S. J.; Bayerl, T. M.; McDermott, D. C.; Adam, G. W.; Rennie, A. R.; Thomas, R. K.; Sackmann, E. Biophysical Journal 1991, 59, 289-294; Bayerl, T. M.; Bloom, M. Biophysical Journal 1990, 58, 357-362), resulting in the solid body dominating important movement properties of the bilayer (e.g. diffusion of the lipids in the plane of the bilayer (M. Hetzer, S. Heinz, S. Grage, T. M. Bayerl, Langmuir, 1998, 14, 982-984)). This technique was also used to immobilize proteins, which were reconstituted in liposomes, on untreated, planar surfaces by means of fusion (J. Salafsky, J. T. Groves, S. G. Boxer, Biochemistry, 1996, 35, 14773-14781). The differences as compared with natural cell membranes which resulted from this, and the small distance between the bilayer and the solid body impeded the functional immobilization of integral membrane proteins, in particular. Thus, more recent research studies are concerned, in particular, with increasing the distance between the bilayer and the solid body in order to uncouple the bilayer dynamically and to enable the integral membrane proteins in the bilayer to be at a sufficiently large distance from the solid-body surface, which frequently has a denaturing effect. This is a prerequisite, for example, for using ion channels in the bilayer as a biosensor.

Current strategies for achieving this aim consist, in particular, in binding lipid molecules chemically or physically on the solid-body surface by way of hydrophilic spacers (“distancing elements”) (B. A. Cornell, V. L. Braach-Maksvytis, L. G. King, P. D. Osman, B. Raguse, L. Wieczorek, R. J. Pace, Nature, 1997, 387, 580; S. Lingler, I. Rubinstein, W. Knoll, A. Offenhäuser, Langmuir, 1997, 13, 7085; H. J. Galla, C. Steinem, K. Rheis, Patent DE 19607279), with the hydrophilic spacer serving to uncouple the bilayer, which is formed from further lipid molecules, from the substrate by the bound lipids acting as “anchors” and thereby specifying the maximum distance between the bilayer and the surface by the length of the hydrophilic spacer. In this connection, the lipid molecules can be bonded to polymeric, oligomeric or low-molecular-weight spacers. Disadvantages are the high preparative input involved in these strategies and the dependence of the properties of the bilayer on the lateral density of the anchor molecules, on the ability of the hydrophilic spacer material to be hydrated and on the dynamic properties of the spacer itself.

The use of solid-body surfaces which have been modified with a hydrophilic polymer (e.g. dextran) and subsequently coated with a bilayer (M. Kuhner, E. Sackmann, Langmuir, 1996, 12 (20), 4866; G. Elender, M. Kuhner, E. Sackmann, Biosens. Bioelectron., 1996, 11, 565; E. Sackmann, Science, 1996, 271, 43; E. Györvary, B. Wetzer, U. B. Sieytr, A. Sinner, A. Offenhäusser, W. Knoll, Langmuir 1999, 15, 1337) therefore represents a further step towards achieving a bilayer which is to a large extent uncoupled from the solid-body surface. The studies, which are described below and which use polyelectrolyte monolayers or multilayers as hydrophilic polymers for uncoupling the bilayer from a planar solid-body surface, are also to be seen in this context. J. Majewski, J. Y. Wong, C. K. Park, M. Seitz, J. N. Israelachvili, G. S. Smith, Biophysical Journal 1998, 75, 2363 describe the possibility of subsequently uncoupling an already existing solid body-supported lipid membrane on a planar quartz substrate from the solid-body surface by treating with a branched cationic polyelectrolyte, in connection with which, however, a stepwise construction, or a direct fusion, of the vesicles on the polycation layer did not lead to a defined bilayer. J. Y. Wong, J. Majewski, M. Seitz, C. K. Park, J. N. Israelachvili, G. S. Smith, Biophysical Journal 1999, 77, 1445 show that a defined lipid membrane can only be obtained on a previously dried PEI (polyethylenimine) layer. On the other hand, success was achieved (U. Sohling, A. J. Schouten, Langmuir 1996, 12, 3912) in constructing a solid body-supported lipid membrane on PEI-modified surfaces in the case of negatively charged lipids. B. Lindholm-Sethson, Langmuir 1996, 12, 3305 reports that defined bilayers composed of partially negatively charged lipids are also obtained on anionic PSS (Na polystyrenesulfonate) surfaces as a result of using Ca²⁺ bridges. Finally, B. Lindholm-Sethson, J. C. Gonzales, G. Puu, Langmuir 1998, 14, 6705 show that cytochrome C oxidase-containing proteoliposomes can be functionally immobilized on polyelectrolyte multilayers.

However, all these studies were carried out on planar substrates, resulting in only a small total surface being available and thereby making chromatographic applications, for example, impossible and other applications more difficult.

Other approaches for preparing polymer-supported lipid membranes make use of what are termed film balance techniques such as Langmuir-Blodgett and Langmuir-Schäfer and are therefore essentially restricted to planar surfaces and likewise unsuitable for certain applications. An example of this is described in H. Hillebrandt, G. Wiegand, M. Tanaka, E. Sackmann, Langmuir 1999, 15, 8451.

Dispensing with spacer elements in this strategy enables the bilayer to be uncoupled from the solid-body surface to the highest degree possible and, at the same time, it is possible, in this way, for integral proteins to be embedded in the membrane without coming directly into contact with the solid body. The crucial problem of this method is the stability of the bilayer on the polymer surface, which is an important criterion for applications in bioanalysis and biosensor technology. For this reason, external forces (e.g. flux forces arising from a medium which is flowing past) readily lead, in this strategy, to parts of the bilayer becoming detached.

Other strategies are based on the formation of lipid monolayers (termed monolayers in that which follows) on solid-body surfaces which have previously been hydrophobized by means of alkylsilane monolayers or mercaptan monolayers. The disadvantage in this case is obvious: no true uncoupling from the substrate surface takes place, thereby significantly altering the dynamics and molecular arrangement of the monolayers as compared with those of a bilayer (Linseisen, F. M.; Hetzer, M.; Brumm, T.; Bayerl, T. M. Biophysical Journal 1997, 72, 1659-1667) and impeding, or even preventing, the incorporation of transmembrane proteins. Thus, C. Steinem et al. (Steinem, C.; Janshoff, A.; Galla, H. J.; Sieber, M., Bioelectrochem. Bioenerg. 1997, 42 (2), 213-220) showed that the proteoliposomes probably do not fuse to form the lipid bilayer but, instead, only become attached, or fuse partially, such that, ultimately, immobilized vesicles are present on the surface. Either older studies relating to this topic did not investigate this possibility or else their functional data which have been presented also allow of the interpretation that there is no direct incorporation of the protein in a lipid bilayer and that the enzyme activity results from the vesicular structures.

The invention now sets itself the object of making available a method which is to a large extent universally applicable, which avoids the above-described deficiencies in the conventional strategies for immobilizing lipid layers and which makes it possible, in a preparatively simple manner, to uncouple the lipid layers, in particular the double lipid layers, from the membrane [sic]. In the novel method, the solid-body surface is to be optimized such that, in association with exhibiting a high degree of stability towards external forces, the solid body-supported lipid layers exhibit properties which are as close as possible to the properties of natural membrane systems.

This object is achieved by means of a method having the features given in claim 1. Preferred embodiments of the method are presented in claims 2 to 24. Claims 25 to 27 relate to an appropriately modified solid-body surface, to an immobilized lipid layer and, respectively, to a kit for preparing immobilized lipid layers. The wording of all the claims is hereby incorporated into the description by reference.

In the method according to the invention, solid-body surfaces are first of all modified such that they offer optimal conditions for the gentle immobilization of a lipid layer, in particular double lipid layer or lipid membrane, which has been applied over the modified surface. In a second step of the method, lipid layers, for example membranes from native cells, are then immobilized on the modified surface such that the properties of the immobilized lipid layers essentially correspond to those of lipid layers which have not been immobilized. In this connection, the lipid layers are preferably immobilized by means of the fusion of vesicles from buffer solutions.

The abovementioned properties of the lipid layers firstly relate to the diffusivity of the lipid layer. A lipid layer, in particular a double lipid layer, which has not been immobilized is characterized by the fact that the individual lipids and other components, for example proteins, can move relatively freely within the layer and, in the case of a double layer, between the two layers as well. In conventional immobilization methods, this diffusivity of the lipid layers is extremely reduced. In the case of a double layer, the opportunity for the lipids to move is markedly retarded, in particular, in the lipid layer which is assigned to the surface of the substrate. This leads to a drastic change in the properties of the conventionally immobilized membrane. In the immobilization method according to the invention, the opportunity for movement within the lipid layer is not restricted, which means that this characteristic property is retained in the lipid layers which have been immobilized in accordance with the invention.

Furthermore, as a result of the modification of the solid-body surface, the immobilization method according to the invention achieves a “soft” surface which, in the first place, leaves the lipid layer which is immobilized on it to a large extent unaffected and, in the second place, also ensures an adequate distance between the solid-body surface and the lipid layer such that the solid-body surface does not exert any denaturing effects on the proteins, in particular enzymes, which the lipid layer may possibly contain. This thereby ensures that the activities of proteins which are embedded in the lipid layer are preserved.

This feature of the invention is exceptionally important for the different possible applications of the invention. Immobilized membranes which retain their native properties, that is, for example, enzymic activities, or else channel activities, are exceptionally important in biosensor technology, in particular. When conventional methods were used, it was not possible to immobilize the membranes in this way while retaining their native properties. The invention therefore opens up entirely new opportunities in biosensor technology and, naturally, in other quite different fields as well, for example in diagnostics or research generally.

It was not possible to imagine, from the present state of knowledge, that such “dynamically” immobilized membranes would exhibit adequate stability on the given support. On the contrary, the skilled person had assumed that actual fixing of the membrane, in particular by means of the linkers which have already been mentioned above, was necessary for immobilization so as to ensure that the membrane was not immediately detached from the support once again. The other known possibility for obtaining a lipid layer which was sufficiently stably immobilized was to have a relatively small distance between the lipid layer and the support, as is achieved by means of an abovementioned immobilization using dextran or polyelectrolytes. The skilled person had consequently assumed that an immobilized membrane either had to be fixed covalently, with the disadvantage of the lack of diffusivity within the membrane, or that the membrane had to be brought very close to the support, in connection with which, however, the support then exerted denaturing effects on the lipid layer or on the proteins which it contained.

The results achieved by the inventors using membranes which have been modified in accordance with the invention are therefore very surprising since it turned out that the “dynamically” immobilized lipid layers are so stably immobilized on their supports that they are suitable for all conceivable applications. The lipid layers which are immobilized in accordance with the invention are at a distance from the support which is sufficient to ensure that the support does not exert any denaturing effects. In addition to this, linkers which would fix the membrane at distinct sites are avoided. This thereby ensures that a lipid layer which has been immobilized in accordance with the invention retains its dynamic properties, in particular the diffusivity of the different lipid layer components and activities of other possible layer components.

In the description which follows, use is made of some specialist terms from biophysics and from surface chemistry and polymer chemistry which are briefly explained below. For a detailed explanation, the reader is referred to the specialist literature (A. Ulman “An Introduction to Ultrathin Organic Films”, Academic Press, Inc., 1991; Albert L. Lehninger “Prinzipien der Biochemie [Principles of Biochemistry]” Walter de Gruyter 1987; R. W. Armstrong, U. P. Strauss “Polyelectrolytes” in “Enzyclopedia of Polymer Science and Technology”, Eds. A. Klingsber, R. M. Piccinne, A. Salvatore, John Wiley and Sons, 1969, Volume 10, 781; M. Mandel “Polyelectrolytes” in “Enzyclopedia of Polymer Science and Technology”, Eds. N. M. Bikales, J. Conrad, A. Ruks, John Wiley and Sons, 1988, Volume 11, 739; H. G. M. van de Steg, M. A. Cohen Stuart, A. de Keizer, B. H. Bijsterbosch, Langmuir, 1992, 8, 2538).

The “polyions” which are described in the following text describe, in a general manner, polymers which carry ionic and/or ionizable functionalities either in the side chains and/or along the main chain. In that which follows, “polyions” are to be understood as meaning the molecule classes “polyelectrolytes”, “polyampholytes” and “polyzwitterions”.

“Polyelectrolytes” are polymers which have incorporated ionic or ionizable groups in the main chain or side chain. In the sense which is used below, “polyelectrolytes” can also be copolymers composed of ionic/ionizable and nonionic monomer units. Polyelectrolytes can be present either in anionic or cationic form. Examples of anionic polyelectrolytes are poly(styrenesulfonic acid), polyvinyl(sulfonate), poly(acrylic acid), dextran sulfate, PAMAM dendrimers (poly(amidoamines), carboxyl-terminated, half generation) and carboxycellulose. Examples of more complex forms of anionic polyelectrolytes are deoxyribonucleic acids (DNA) and ribonucleic acids (RNA). Examples of cationic polyelectrolytes are poly(allylamine hydrochloride), poly(vinylamine), poly(ethyleneimine), poly(diallylammonium chloride), PAMAM dendrimers (amino-terminated, full generation) and poly(2-vinylpyridine). Examples of ionic copolymers are poly(acrylic acid-co-acrylamide) and poly(diallylammonium chloride-co-acrylamide). It is evident from this brief list that carboxyl, sulfate, sulfonate, phosphate and phosphonate groups are typical functional groups of anionic polyelectrolytes. Typical cationic functionalities are primary, secondary, tertiary and quaternary amine groups and also R₃S(+) groups.

Polyampholytes, which carry ionizable functional groups in the main chain or side chain and whose net charge state depends on the pH of the solution, are polymers which are related to the polyelectrolyte group. In the general sense, “polyampholytes” are also to be understood as meaning proteins and enzymes.

Polyzwitterions, which carry permanent anionic and cationic charges in the main chain or side chain, represent another group of ionic polymers.

The terms “bilayer”, “lipid membrane” and “lipid bilayer”, which are used in the following text are synonymous and refer to a double lipid layer which consists of a hydrophobic internal region and a hydrophilic external region and which arises spontaneously, for example, in connection with the self-organization of natural and synthetic lipids or lipid-like substances in an aqueous phase, or which can be generated by means of transfer techniques (Langmuir-Blodgett technique).

The term “vesicle” refers to unilamellar and multilamellar aggregate forms which lipids and lipid-like substances form spontaneously on swelling in aqueous phase or form under external influence, for example as a result of ultrasound treatment or as a result of high-pressure filtration (extrusion).

The term “substrates”, which is used in the following text, refers to solid bodies which are insoluble in aqueous solution, which are composed of organic or inorganic material and which, after optimization, are used as surfaces (solid-body surfaces) which are sufficiently solid for supporting the lipid layers.

The invention describes a novel method for optimizing the surface properties of pulverulent or particulate substrates of any arbitrary geometry with the aim of as far as possible approximating the properties of the lipid layers, which are to be deposited on them, to those of a natural membrane and, at the same time, enabling integral membrane proteins to be immobilized without any significant loss in their activity.

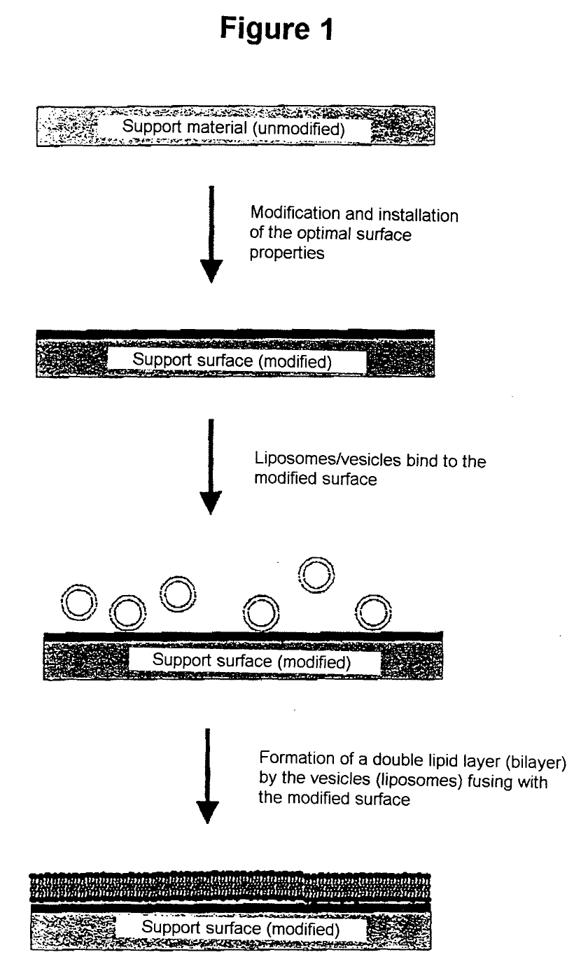

The method according to the invention can be subdivided into two procedural steps. These are, in the first place, (a) modifying the solid-body surface (substrate surface) with molecules in order to form an essentially hydrophilic surface area and (b) depositing the lipid layers on the modified solid-body surface. These procedural steps are illustrated diagrammatically in FIG. 1, which does not show the pulverulent character of the solid body. The first procedural step results in a suitable modification of the substrate surface, which modification ensures that the lipid layer, which is to be deposited in the second procedural step, is to a large extent uncoupled from the substrate while, at the same time, exhibiting a high degree of morphological integrity and stability. In the method, the use of spacer elements which are covalently bonded to the substrate is avoided. The immobilized lipid layer is consequently freely, i.e. without any punctate bonding to the substrate, immobilized on the modified surface.

By means of modifying the surface in accordance with procedural step (a), it is possible, in particular, to form a hydrophilic surface which carries ionic groups, i.e. preferably functional groups, which dissociate in an aqueous environment.

In addition to this, the solid body-supported lipid layer which is formed is very similar to the properties of its natural (solid body-independent) analog (e.g. the lipid vesicle in the case of pure lipids or the biomembrane in the case of natural lipid mixtures with proteins, in particular transmembrane proteins).

Essentially two steps are required for modifying the solid-body surface (procedural step (a)). First of all (aa), the solid-body surface is functionalized, i.e. functional groups, in particular chemically functional groups, are fixed on the surface. In another step (ab), interacting molecules are adsorbed/chemisorbed on the functionalized surface. In addition, further molecules can be adsorbed or chemisorbed in a third step (ac). These molecules can be identical to the molecules employed in step (ab); alternatively, it is possible to use a different molecular species for this further step. What is more, the modification can also include still further appropriate substeps. This can be of value when the distance between the lipid layer and the solid-body surface is to be made particularly large. However, a modification which is performed in only a few steps is frequently particularly advantageous and preferable with regard to the preparative input as well.

The appropriate molecules can be applied, for example, by means of deposition from a solution or from the gas phase.

The surface can also be modified in one step, particularly when a suitable surface-modifying material is commercially available.

Appropriately modifying the solid-body surface (procedural step (a)) results in a hydrophilic surface being formed on the substrate. This thereby enables a lipid layer, in particular a bilayer, to form spontaneously on the modified surface (procedural step (b)). This can preferably be achieved by means of a conventional vesicle fusion on the surface.

After the two procedural steps (a) and (b), i.e. modification of the surface and deposition or immobilization of the lipid layer, have been performed, the result is a stable, solid body-supported lipid layer, preferably a bilayer or a solid body-supported biomembrane having the enzymic activity of the proteins which are immobilized in it. In this connection, the topology of the lipid layer or bilayer surface or membrane surface is to a large extent predetermined by that of the substrate, that is the solid-body surface.

The method according to the invention is consequently preferably characterized in that inorganic and/or organic solid-body surfaces are modified chemically and/or physically such that

a) the surface properties of the support material (substrate) can be influenced and modulated selectively;

b) it is made possible to apply a lipid layer to the modified solid-body surface;

c) the lipid layer is bound to the modified solid-body surface by means of physical and/or chemical forces such that it surrounds this surface completely without, however, the diffusion of the lipid layer constituents within the layer being significantly impaired;

d) the properties of the immobilized lipid layer, in particular a lipid membrane, are as close as possible to the properties of the lipid layer which has not been immobilized or of the natural lipid membrane.

Examples of criteria for comparing the properties of the immobilized lipid layer which is obtained by the method according to the invention with those of its solid body-free analogs are measurements of the phase transition temperature by means of differential microcalorimetry (DSC), measurement of fluidity and mobility by means of solid body nuclear resonance (NMR) or determination of the functionality or activity of immobilized membrane proteins.

In the preferred first substep for modifying the solid-body surface in accordance with the method according to the invention (substep (aa)), functional groups are introduced on the solid-body surface. In a preferred embodiment, this takes place by means of an amino-functionalization, epoxyfunctionalization, haloalkyl-functionalization and/or thiofunctionalization. It is effected by the solid-body surface being treated with correspondingly functional molecules. For this purpose, the molecules which contain the functional groups are preferably dissolved in a solution, for example in an aqueous solution, and the surface material is added to this solution or the solution is applied to the surface material. The incubation can take place, for example, at room temperature over a period of some hours. It is naturally also possible to select other reaction conditions depending on the material which is chosen. Furthermore, the functionalities which are listed only represent examples which a skilled person can extend with additional suitable possibilities.

In a particularly preferred embodiment of the method according to the invention, silanes, which carry an appropriate functionality, are used for the functionalization. Monofunctional, difunctional or trifunctional silanes are particularly suitable for this purpose. Consequently, the silanes can therefore, in this connection, be aminofunctional, epoxyfunctional, haloalkylfunctional and/or thiofunctional silanes.

An example of a particularly suitable aminofunctional silane is N-(2-aminoethyl)-3-aminopropyltrimethoxy-silane (EDA). Suitable epoxyfunctional, haloalkylfunctional and thiofunctional silanes are [3-(2,3-epoxypropoxy)propyl]trimethoxysilane (EPOXY), [3-iodopropyl]trimethoxysilane and, respectively, [3-thiopropyl]trimethoxysilane.

In another preferred embodiment, mercaptans and/or disulfides are used for the functionalization. Functionalization with mercaptans is to be preferred for metallic solid-body surfaces, in particular. Alkyl disulfides are particularly suitable disulfides. The mercaptans and disulfides advantageously carry functional groups.

Mercaptans of differing functionality can be used, in a manner corresponding to that in the case of the abovementioned silanes.

In a particularly preferred embodiment, the aminofunctional mercaptan employed is cysteamine hydrochloride and/or cysteamine. It is naturally also possible to successfully employ other mercaptans, for example epoxyfunctional or haloalkylfunctional mercaptans as well.

It is also possible to use other functional molecules for functionalizing the solid-body surface. A particularly suitable example of this is polyethylenimine (PEI), which imparts aminofunctions to the surface.

As the second preferred substep in modifying the solid-body surface (substep (ab)), interacting molecules are absorbed on the functionalized surface. This absorption on the functionalized surface takes place, in particular, by means of interaction with the functional groups which were applied to the surface in the first substep. The absorption can also be what is termed a chemisorption, in which covalent bonds are formed between the interacting molecules and the functional groups which have been attached to the surface. However, it can also be a matter of other interactions between the interacting molecules and the functionalized surface; for example, electrostatic interactions and also van der Waals' forces come into consideration for this purpose.

In a preferred embodiment of the method according to the invention, the interacting molecules employed are polymers. These polymers can also be biopolymers. It is naturally also possible to use monomers. Suitable polymers are, in particular, polyelectrolytes, polyampholytes and/or polyzwitterions. The polyelectrolytes employed are preferably anionic polyelectrolytes. As is known, the polyampholytes also include proteins, which may be very suitable for this substep in modifying the solid-body surface.

Some examples of polyanions or anionic polyelectrolytes which are particularly suitable are polysulfates, polysulfonates, polycarboxylates, polyphosphates and their free acids, polystyrenesulfonic acid, polystyrenesulfonate (PSS), PAMAM dendrimers (carboxylterminated, half generation), polyacrylic acid, polyacrylate, polymethacrylic acid, polymethacrylate, dextran sulfate, deoxyribonucleic acid and ribonucleic acid. Examples of suitable polycations are polyamines and their salts, polyethylenimine, polyallylamine, PAMAM dendrimers (amino-terminated, full generation), polyvinylamine, polylysine, poly(vinylpyridine) and their salts, and also poly(diallyldimethylammonium chloride).

Bovine serum albumin (BSA) is an example of a suitable protein which can be used, in accordance with the invention, as an interacting molecule. A large number of other proteins, in particular enzymes as well, are naturally also suitable.

In another preferred embodiment of the invention, the interacting (reactive) molecules employed are substances which enter into covalent interactions with the functionalized surface, with what is termed a chemisorption consequently taking place. In this connection, particular preference is given to aminoreactive molecules which are used as interacting molecules for an amino-functionalized surface.

Particularly suitable examples of this are the polymers poly(styrene-co-maleic anhydride) (PSPMA) and poly(ethylene-co-maleic anhydride) (PEPMA). Other suitable amino reactive substances are 3,3′,4,4′, benzophenonetetracarboxylic dianhydride and 3,3′,4,4′-biphenyltetracarboxylic dianhydride.

In addition to these aminoreactive substances or polymers, the invention also encompasses other interacting or reactive molecules which will be evident, without difficulty, to a skilled person from what has been said thus far.

In another preferred embodiment according to the invention, the surface is provided with epoxyfunctional groups in the first substep of the modification and treated with correspondingly suitable epoxyreactive molecules in the second substep of the modification. Examples of epoxyreactive polymers are polyethylenimine, polyallylamine, polyallylamine hydrochloride, polyvinylamine, poly(ethylene glycol), polyvinyl alcohol and dextran. As is apparent, polyamines can be applied both as a functionalizing layer in a single-step process and as an interacting layer in two-step processes. This is always advantageous when the original properties of the solid body are to be changed, in the first step, by means of covalently reacting the surface functionalities.

It is also suitable to use proteins, in particular enzymes, as interacting molecules in the case of an epoxyfunctionalized surface. Other suitable epoxyreactive substances are sodium thiosulfate, ethylene diamine hydrochloride and taurine.

By means of using charged molecules, in particular polymers, when modifying the surface, it is possible to optimize the surface properties so as to minimize the undesirable charge effects which are intrinsic to the surface material. The reagents which are suitable for this purpose should be selected appropriately depending on the desired goal of the experiment.

In a particularly preferred embodiment of the method according to the invention, further molecules, which, in particular, occasion an additional functionality of the surface, are inserted between the solid-body surface and the lipid layer which is to be applied. Thus, it is possible, for example, to integrate dyes, in particular fluorescent dyes, nanoparticles or biomolecules, such as proteins for example, in particular enzymes, into the modified surface layer. This makes it possible to incorporate probe molecules into the system of solid body-supported lipid layers or lipid membranes. Furthermore, it is possible, in this way, to incorporate chemically or photochemically reactive functionalities into the modified surface, something which can be used, for example, for covalently immobilizing transmembrane proteins in an otherwise fluid, i.e. essentially liquid, lipid membrane. These functionalities which are to be additionally introduced can, for example, be covalently bound or else bound by way of other interactions with the solid-body surface.

In principle, all pulverulent support materials which are known in the prior art are suitable for use as solid-body surfaces, that is as supports for the lipid layer or membrane which is to be immobilized. Advantageously, these support materials can also be porous in order to provide an even larger surface.

Silicate surfaces or silicate particles are particularly suitable for use as solid-body surfaces. It is furthermore possible to successfully employ porous or nonporous aluminates, borates or zeolites. Colloidal solutions of precious metals, metals, etc., are also suitable. In a particularly preferred embodiment, these solid-body surfaces employed are pulverulent and, preferably, porous polymer surfaces.

In another preferred embodiment, the solid-body surfaces employed are magnetic supports, for example polymer microspheres containing a magnetic core. It is furthermore possible to advantageously employ core-shell polymer particles as the support material.

In a preferred embodiment of the method according to the invention, materials which are customarily used as packing material in chromatographic columns are employed as the solid-body surface material.

In addition to this, it is also possible to use, as surface materials, films which are composed of pulverulent metals, semiconductors, precious metals and/or polymers which are applied to support materials which are, in particular, to a large extent planar, or which can be applied to these materials. These latter support materials can, for example, be substrates made of paper, glass, plastic or the like to which the solid bodies are bonded in a suitable manner, in particular by means of gluing or fusing.

The modification of the solid-body surface in accordance with the method according to the invention makes the surface suitable for immobilizing very different lipid layers. This modified solid-body surface is very particularly suitable for immobilizing double lipid layers and, in particular, for immobilizing lipid membranes. Double lipid layers or lipid membranes are also of particular interest for the diverse applications of the invention in research, diagnostics and, in particular, biosensor technology, with it being possible to use the native properties of such layers as models for natural systems.

According to the invention, the lipid layers are composed of substances from the substance classes represented by lipids, lipid derivatives and lipid-like and/or lipid-analogous substances. In addition, the lipid layers can also contain peptides, proteins, nucleic acids, ionic or nonionic surfactants and/or polymers. The presence of these additional components in the immobilized lipid layers makes it possible to copy natural systems by, for example, channel proteins or enzymes being contained in the lipid layers. These additional components may be present on the surface of the lipid layer or embedded in the lipid layer, that is be present integrally in the lipid layer. Furthermore, the additional components can extend through the entire layer or membrane, that is extend transmembranally (spanning the layer). These proteins can be enzymes, receptors and/or ligands, with the receptors and/or ligands preferably being at least partially aligned on the surface of the lipid layer which is facing away from the support. Furthermore, the lipid membrane can be constructed from proteoliposomes.

In a particularly preferred embodiment of the method according to the invention, at least some of the lipid layers which are to be immobilized are membrane fragments derived from natural cells. It is naturally also possible for corresponding artificial lipid layers or membranes to be assembled in vitro from various components, thereby providing a corresponding membrane model. However, in a particularly preferred manner, membranes are isolated from natural cells and deposited on the surfaces which have been modified in accordance with the invention.

In another embodiment of the invention, the immobilized lipid layer, in particular the lipid membrane, contains proteins which are preferably arranged transmembranally and/or integrally, with these proteins being able to bind, or having bound, water-soluble proteins peripherally.

The lipid layers are preferably deposited on the modified solid-body surface by means of lipid vesicles being fused in a conventional manner. These vesicles can be vesicles of a defined composition which are composed, for example, of phospholipids. On the other hand, it is naturally also possible to isolate membrane vesicles from natural material and to use them in the sense of the invention [sic] according to the invention. Membrane vesicles which are obtained from the sarcoplasmic reticulum, for example, are particularly suitable for this purpose. The vesicles are prepared using methods with which the skilled person is familiar. A major advantage of the method according to the invention is to be seen in the fact that such vesicles only have to be brought into contact with the surface which has been modified in accordance with the invention for the vesicles to fuse spontaneously with each other and form a lipid layer. For this reason, the method according to the invention represents a system which is extremely easy to operate and which only requires a small degree of preparative input.

The invention furthermore encompasses a modified solid-body surface as can be prepared in accordance with the method according to the invention. In addition, it encompasses a lipid layer which has been appropriately immobilized on a modified solid-body surface. The reader is referred to the above description with regard to the features possessed by the modified solid-body surface or the immobilized lipid layer.

Finally, the invention encompasses a kit for preparing immobilized lipid layers on solid-body surfaces. Such a kit comprises at least one solid-body surface which has been modified in accordance with the above description. In another variant, the kit comprises reagents for preparing an appropriately modified solid-body surface. In addition, it is possible to envisage that the kit would additionally contain reagents for depositing lipid layers on a modified solid-body surface. However, it may also be preferable for these reagents to be prepared by the given user himself. This applies, in particular, when it is a matter of isolating membranes from natural systems and then immobilizing these membranes in accordance with the invention.

The above-described features, and additional features, of the invention ensue from the following description of examples taken in combination with the figures and the subclaims. In this connection, the different features can in each case be realized on their own or in combination with each other.

The figures depict the following:

FIG. 1 shows a diagram of the procedure in the method according to the invention.

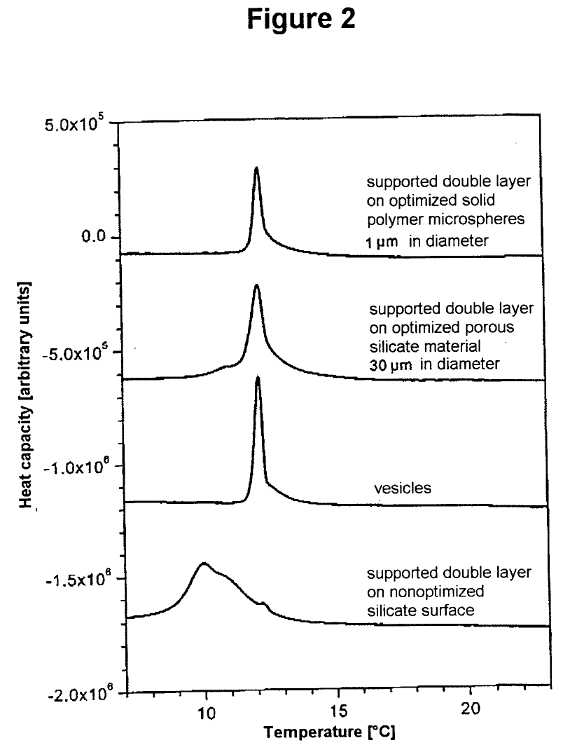

FIG. 2 shows DSC plots of dielaidoylphosphatidylcholine (DEPC)-coated, porous silicate particles which do or do not possess optimized surface as compared with natural DEPC vesicles which lack a support material.

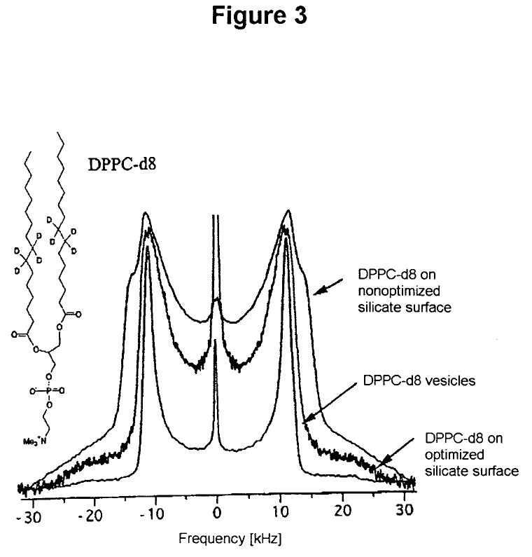

FIG. 3 shows deuterium (²H)-NMR spectra of non-porous silicate particles which are coated with selectively chain-deuterated dipalmitoylphosphatidylcholine (DPPC)-d8 (7,7′,8,8′-D₂) and which do or do not possess an optimized surface, as compared with natural DPPC-d8 (7,7′,8,8′) vesicles which lack any support material.

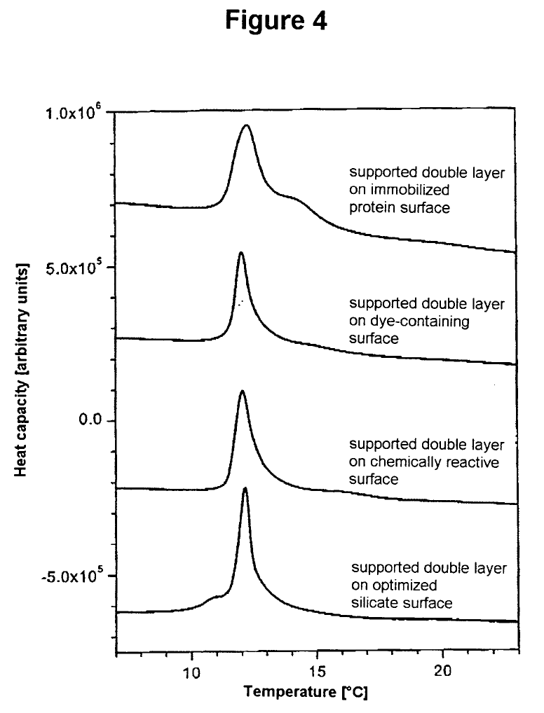

FIG. 4 shows DSC measurements of DEPC-coated, porous silicate particles on complex, optimized surfaces.

### EXAMPLES

1. Modifying the Solid-Body Surface

1.1. Functionalizing the Solid-Body Surface

1.1.1 Amino-Functionalizing Pulverulent and Porous Silicate Surfaces with N-(2-aminoethyl)-3-aminopropyltrimethoxysilane (EDA)

A silane solution consisting of 1.05 ml of N-(2-aminoethyl)-3-aminopropyltrimethoxysilane (EDA) and 27 μl of concentrated acetic acid in 100 ml of deionized water was prepared freshly. After 5 minutes, 5 g of a porous silicate material (Nucleosil 4000-30 from Macherey-Nagel, Düren) were added to the silane solution and suspended by shaking the mixture. This dispersion was rotated slowly for 3 hours and, after that, the silicate material was sedimented and washed three times with deionized water. The success of the silanization was documented by means of infrared spectroscopy in diffuse reflection (DRIFT) using the dried silicate material.

1.1.2. Amino-Functionalizing Pulverulent and Porous Silicate Surfaces with Polyethylenimine (PEI)

5 g of a porous silicate material (Nucleosil 4000-10 from Macherey-Nagel, Düren) were added to a polyethyleneimine (PEI) solution consisting of 250 mg of PEI (50% solution in water, Aldrich, Steinheim) in 50 ml of deionized water, and the mixture was rotated slowly for 3 hours. After that, the silicate material was sedimented and washed three times with deionized water. The success of the silanization was documented by means of infrared spectroscopy in diffuse reflection (DRIFT) using the dried silicate material.

1.1.3. Thio-Functionalizing Pulverulent and Porous Silicate Surfaces

A silane solution consisting of 1 ml of mercaptopropyl-trimethoxysilane (THIO) in 50 ml of 2-propanol was prepared freshly. After 5 minutes, 5 g of a porous silicate material (Nucleosil 4000-30 from Macherey-Nagel, Düren) were added to the silane solution and suspended by shaking the mixture. This dispersion was rotated slowly for three hours, after which the silicate material was sedimented and the supernatant was removed. The material was firstly dried at 80° C. and then after-baked at 100° C. for one hour. After that, it was washed three times with 2-propanol. The success of the silanization was documented by means of DRIFT.

1.1.4. Epoxy-Functionalizing Pulverulent and Porous Silicate Surfaces

A silane solution consisting of 2 ml of [3-(2,3-epoxypropoxy)propyl]trimethoxysilane (GPS) in 100 ml of 2-propanol was prepared freshly. After 5 minutes, 10 g of a porous silicate material (Nucleosil 4000-30 from Macherey-Nagel, Düren) were added to the silane solution and suspended by shaking the mixture. This dispersion was rotated slowly for three hours, after which the silicate material was sedimented and the supernatant was removed. The material was firstly dried at 80° C. and then after-baked at 100° C. for one hour. After that, it was washed three times with 2-propanol. The success of the silanization was documented by means of DRIFT.

1.1.5. Amino-Functionalizing Magnetic Particles with Polyethylenimine (PEI)

2 ml of a suspension of magnetic polystyrene particles (Dynabeads® M-280, tosyl-activated, from Dynal, Oslo, Norway) are washed with 0.1 M NaCl in water. 5 ml of a polyethylenimine (PEI) solution, consisting of 250 mg of PEI (50% solution in water, Aldrich, Steinheim) in 50 ml of 0.1 M NaCl in deionized water, are added to the resulting suspension and the mixture is rotated slowly for three hours. After that, the particles are separated off using a magnet and washed three times with 0.1 M NaCl in water. The success of the reaction is determined by means of titration.

1.2. Adsorption/Chemisorption of Interacting Molecules

1.2.1. Adsorbing Na-polystyrenesulfonate (PSS) on EDA-functionalized Silicate Surfaces

1 g of a silicate material which had been amino-functionalized as described in Example 1.1.1. was added to a Na-polystyrenesulfonate (PSS) solution consisting of 12.5 mg of PSS (Aldrich, Steinheim) in 25 ml of deionized water and the mixture was shaken for three hours. After that, the silicate material was sedimented and washed three times with deionized water. The success of the adsorption was documented by means of DRIFT and the decrease in the concentration of PSS in the solution.

1.2.2. Adsorbing Na-polystyrenesulfonate (PSS) on PEI-Functionalized Silicate Surfaces

1 g of a silicate material which had been amino-functionalized as described in Example 1.1.2. was added to a Na-polystyrenesulfonate (PSS) solution consisting of 25 mg of PSS (Aldrich, Steinheim) in 25 ml of deionized water, and the mixture was shaken for 3 hours. After that, the silicate material was sedimented and washed three times with deionized water. The success of the adsorption was documented by means of DRIFT and the decrease in the concentration of PSS in the solution.

1.2.3. Adsorbing Na-polystyrenesulfonate (PSS) on Amino-Functionalized Pulverulent Polymer Surfaces

4 ml of a latex dispersion, consisting of amino-functionalized polystyrene latices (K2-080, Prolabo, France), were added to a solution consisting of 3 ml of 0.1 molar Na-polystyrenesulfonate (Aldrich, Steinheim) solution and 5 ml of a 4.8 molar NaCl solution, and the mixture was shaken overnight. After that, the modified polymer material was separated off by centrifugation. For washing, the sedimented polymer material was resuspended in 10 ml of deionized water, shaken and then centrifuged once again. This washing step was repeated a total of four times and the polymer material was finally resuspended in 4 ml of deionized water. The success of the adsorption was documented by titrating the surface charges.

1.2.4. Chemisorbing Poly(Styrene-Co-Maleic Anhydride) (PSPMA) on EDA-Functionalized Silicate Surfaces

5 g of a silicate material which had been amino-functionalized as described in Example 1.1.1. were added to a poly(styrene-co-maleic anhydride) (PSPMA) solution consisting of 250 mg of PSPMA (Aldrich, Steinheim) in 200 ml of acetone, and the mixture was shaken for 3 hours. After that, the silicate material was sedimented and washed three times with acetone. The success of the chemisorption was documented by means of DRIFT.

1.2.5. Chemisorbing Poly(Styrene-Co-Maleic Anhydride) (PSPMA) on PEI-Functionalized Silicate Surfaces

5 g of a silicate material which had been amino-functionalized as described in Example 1.1.2. were added to a poly(styrene-co-maleic anhydride) (PSPMA) solution consisting of 250 mg of PSPMA (Aldrich, Steinheim) in 200 ml of acetone, and the mixture was shaken for 3 hours. After that, the silicate material was sedimented and washed three times with acetone. The success of the chemisorption was documented by means of DRIFT.

1.2.6. Adsorbing Bovine Serum Albumen (BSA) on EDA-Functionalized Silicate Surfaces

1 g of an EDA support material which had been functionalized as described in Example 1.1.1. was added to a solution consisting of 220 mg of bovine serum albumen (BSA, from Sigma-Aldrich, Steinheim) in 50 ml of a 25 mM HEPES buffer, pH 7.1 (buffer A), and the mixture was rotated for three hours. After that, the support material was sedimented, washed three times with buffer A and then dried. The success of the coating was documented by means of DRIFT and measuring the decrease in the concentration of BSA in the solution.

1.2.7. Adsorbing Sodium Thiosulfate on Epoxyfunctionalized Silicate Surfaces

In order to prepare an anionic surface, 1 g of a material which had been prepared as described in Example 1.1.4. was rotated for 16 hours in an 0.1 molar solution of sodium thiosulfate. After that, the material was sedimented, washed three times with deionized water and dried at 80° C. The success of the coating was documented by means of DRIFT.

1.2.8. Chemisorbing Polyethylenimine on Epoxyfunctionalized Silicate Surfaces

1 g of a material which had been prepared as described in Example 1.1.4 was added to a polyethylenimine (PEI) solution consisting of 250 mg of PEI (50% solution in water, Aldrich, Steinheim) in 50 ml of deionized water, and the mixture was rotated slowly for three hours. After that, the material was sedimented and washed three times with deionized water. The success of the coating was documented by means of DRIFT.

1.2.9. Adsorbing Polyallylamine Hydrochloride (PAH) on PSS-Functionalized Silicate Surfaces and Further Adsorption of PSS

In order to prepare a modified surface in a multi-step method, 1 g of a silicate material which had been PSS-functionalized as described in Example 1.2.1. was added to a polyallylamine hydrochloride (PAH) solution consisting of 12.5 mg of PAH (Aldrich, Steinheim) in 25 ml of deionized water, and the mixture was shaken for three hours. After that, the silicate material was sedimented and washed three times with deionized water. The success of the adsorption was documented by means of DRIFT. 0.5 g of the resulting PAH-functionalized silicate material was added to a solution consisting of 10 mg of PSS (Aldrich, Steinheim) in 10 ml of deionized water and the mixture was shaken for three hours. After that, the silicate material was sedimented and washed three times with deionized water. The success of the adsorption was documented by means of DRIFT.

1.2.10. Adsorbing Na-Polystyrenesulfonate (PSS) on PEI-Functionalized Magnetic Particles

5 ml of an Na-polystyrenesulfonate (PSS) solution, consisting of 12.5 mg of PSS (Aldrich, Steinheim) in 25 ml of 0.1 M NaCl in deionized water, are added to the suspension prepared as described in Example 1.1.5., and the mixture is shaken for three hours. After that, the particles are separated off and washed three times with 0.1 M NaCl in deionized water. The success of the adsorption is documented by means of titration and by the decrease in the concentration of PSS.

1.3. Inserting Further Functional Molecules into the Modified Surface

1.3.1. Preparing Photoreactive Surfaces on PSS/EDA-Functionalized Silicate Surfaces

1 g of an EDA/PSS support material which had been functionalized as described in Example 1.2.1. was added to a solution consisting of 0.2 g of 3,3′,4,4′-benzophenonetetracarboxylic dianhydride (BPA) in 25 ml of acetone, and the mixture was rotated overnight. After that, the support material was sedimented, washed three times with acetone and dried. The success of the treatment was documented by means of DRIFT.

1.3.2. Preparing Photoreactive Surfaces on EDA-Functionalized Silicate Surfaces

1 g of an EDA support material which had been functionalized as described in Example 1.1.1 was added to a solution consisting of 0.2 g of 3,3′,4,4′-benzophenonetetracarboxylic dianhydride (BPA) in 25 ml of acetone, and the mixture was rotated overnight. After that, the support material was sedimented, washed three times with acetone and dried. The success of the treatment was documented by means of DRIFT.

1.3.3. Preparing Anhyride Surfaces on PSS/EDA-Functionalized Silicate Surfaces

1 g of an EDA/PSS support material which had been functionalized as described in Example 1.2.1. was added to a solution consisting of 0.1 g of 3,3′,4,4′-biphenyltetracarboxylic dianhydride in 25 ml of acetone, and the mixture was rotated overnight. After that, the support material was sedimented, washed three times with acetone and dried.

1.3.4. Preparing Dye Surfaces on PSS/EDA-Functionalized Silicate Surfaces

1 g of an EDA/PSS support material which had been functionalized as described in Example 1.2.1. was added to a solution consisting of 2 mg of fluorescein isothiocyanate (FITC) in 10 ml of ethanol, and the mixture was rotated overnight. After that, the support material was sedimented, washed three times with ethanol and dried.

1.4. Lipid Anchors

1.4.1. Preparing Lipid Anchors from Chemically Reactive Surfaces

1 g of an EDA/PSPMA material which had been prepared as described in Example 1.2.4. was rotated for 16 hours in a solution of 50 mg of dioleoylphosphatidylethanolamine (DOPE) in triethylamine/chloroform. After that, the material was sedimented and washed three times with chloroform. The success of the coating was documented by means of DRIFT.

1.4.2. Preparing Lipid Anchors from Ionic Surfaces

1 g of an EDA/PSS material which had been prepared as described in Example 1.2.1 was rotated for 16 hours in a solution of 50 mg of dioctadecyldimethylammonium bromide (DODAB) in chloroform. After that, the material was sedimented and washed three times with chloroform. The success of the coating was documented by means of DRIFT.

2. Depositing Lipid Layers on the Modified Surfaces

2.1. Preparing Lipid Vesicles for Immobilizing on Pulverulent Surfaces

80 mg of dielaidoylphosphatidylcholine (DEPC) were swollen, at room temperature for half an hour, in 16 ml of coating buffer, consisting of 20 mM HEPES buffer, pH 7.1 containing 30 mM NaCl, and then ultrasonicated for 30 minutes using a probe sonicator (Branson Sonorex). The result was a clear dispersion of vesicles with the vesicles having diameters in the range of 20-80 nm. This was determined by means of conventional dynamic laser light scattering (particle-sizing).

2.2. Immobilizing Lipid Membranes on Optimized Pulverulent Silicate Surfaces

1 g of a porous silicate support which had been optimized as described in Example 1.2.1. was added to 16 ml of a vesicle dispersion which had been prepared as described in Example 2.1, and the mixture was rotated slowly for 30 minutes. After that, the support material was sedimented and washed three times with coating buffer. The success of the coating was documented by means of DSC using the material dispersed in coating buffer (as described in C. Naumann, T. Brumm, T. M. Bayerl, Biophys. J., 1992, 63, 1314) and by means of DRIFT (after having dried the material).

2.3. Immobilizing Lipid Membranes on Optimized Pulverulent Polymer Surfaces

1.3 ml [sic] of a polymer support which had been modified as described in Example 1.2.3. were added to 4 ml of a vesicle dispersion which had been prepared as described in Example 2.1., and the mixture was rotated slowly for 30 minutes. After that, the support material was sedimented and washed three times with coating buffer. The success of the coating was documented by means of DSC using the material dispersed in coating buffer.

2.4. Immobilizing Lipid Membranes on Optimized Pulverulent Surfaces of Mixed Functionality

1 g of a porous silicate support which had been optimized as described in Example 1.3.4. was added to 16 ml of a vesicle dispersion which had been prepared as described in Example 2.1, and the mixture was rotated slowly for 30 minutes. After that, the support material was sedimented and washed three times with coating buffer. The success of the coating was documented by means of DSC, using the material dispersed in coating buffer, and DRIFT (after having dried the material).

2.5. Immobilizing Lipid Membranes on Optimized Pulverulent Surfaces Possessing Lipid Anchors

1 g of a porous silicate support which had been optimized as described in Example 1.4.1. was added to 16 ml of a vesicle dispersion which had been prepared as described in Example 2.1., and the mixture was rotated slowly for 30 minutes. After that, the support material was sedimented and washed three times with coating buffer. The success of the coating was documented by means of DSC, using the material dispersed in coating buffer, and DRIFT (after having dried the material).

2.6. Immobilizing Native Sarcoplasmic Reticulum (SR) Membranes on EDA/PSS-Modified Silicate Surfaces and Measuring the Ca²⁺-ATPase Function

A method developed by W. Hasselbach and M. Makinose (Biochem Z. 1961, 333, 518-528) was used to prepare sarcoplasmic reticulum membrane vesicles (SR vesicles) from the muscle tissue of a rabbit. This dispersion was then converted, by means of ultrasonication treatment, into small, single-shelled vesicles having a diameter of 20-90 nm. 50 mg of a porous silicate support which had been optimized as described in Example 1.2.1. were added to 900 μl of this solution (about 0.5 mg of total protein), and the mixture was incubated at 4° C. for 18 hours, with 100 mM triethanolamine (pH 7.4) and 100 mM NaCl being used as the buffer solution (incubation buffer). After that, the support material was sedimented and washed three times with incubation buffer. The success of the coating was documented by means of DRIFT using the dried material. After the washing in the incubation buffer, the Ca²⁺-ATPase activity on the support material was effected by determining the ATP hydrolysis activity in dependence on the calcium ion concentration, and its inhibition by the specific inhibitor cyclopiazonic acid. This function test verified that a Ca²⁺-ATPase activity which was comparable to the SR vesicle was present on the support material.

2.7. Immobilizing Native Sarcoplasmic Reticulum (SR) Membranes on GPS/PEI-Modified Silicate Surfaces and Measuring the Ca²⁺-ATPase Function

A method developed by W. Hasselbach and M. Makinose (Biochem Z. 1961, 333, 518-528) was used to prepare sarcoplasmic reticulum membrane vesicles (SR vesicles) from the muscle tissue of a rabbit. This dispersion was then converted by ultrasonication treatment, into small, single-shelled vesicles having a diameter of 20-90 nm. 50 mg of a porous silicate support which had been optimized as described in Example 1.2.8. were added to 900 μl of this solution (about 0.5 mg of total protein), and the mixture was incubated at 4° C. for 18 hours, with 100 mM triethanolamine (pH 7.4) and 100 mM NaCl being used as the buffer solution (incubation buffer). After that, the support material was sedimented and washed three times with incubation buffer. The success of the coating was documented by means of DRIFT using the dried material. After the washing in the incubation buffer, the Ca²⁺-ATPase activity on the support material was [lacuna] by determining the ATP hydrolysis activity in dependence on the calcium ion concentration and its inhibition by the specific inhibitor cyclopiazonic acid. This function test verified that a Ca²⁺-ATPase activity which was comparable to the SR vesicle was present on the support material.

3. Properties of the Membranes on Optimized Support Material

3.1. Stability in Flowing Aqueous Medium

The systems described in Examples 2.2. to 2.6 were exposed to a flowing medium (coating buffer as described in Example 2.1 or incubation buffer as described in Example 2.6) in a test bath for a period of 24 hours. In each case equal quantities of support material were removed from the test bath at intervals of 2 hours and dried, after which their coating was analyzed by means of DRIFT. The systems described in Examples 2.2 to 2.5 were additionally analyzed by means of DSC. No measurable decrease in the membrane coating with time was observed either with DRIFT or with DSC.

3.2. Stability Following Drying and Renewed Dispersion

The systems described in Examples 2.2 to 2.5 were dried and the quantity of lipid present on them was documented by means of DRIFT. The samples were then resuspended in coating buffer and washed three times. After this treatment, the sample was dried once again and measured by means of DRIFT. In all cases, it was not possible to detect any change in the quantity of lipid which was present on the support material.

3.3. Stability Following Freezing

The systems described in Examples 2.2 to 2.5 were frozen at −80° C. in the dispersed state and then once again brought to room temperature and dried. Comparative DRIFT measurements carried out before and after the freezing showed that the quantities of lipid on the support material were unchanged.

3.4. Stability of the Enzymic Activity of SR-Coated Support Material

After having been prepared, the system described in Example 2.6 was stored at −80° C. for a period of 3 months. Samples were removed at intervals of 1 month and their Ca²⁺-ATPase activity was analyzed using the method described in 2.6. After 2 months, the activity had fallen to approx. 70% of the original value (as measured immediately after the support material had been prepared and washed). It was not possible to measure any Ca²⁺-ATPase activity in the supernatant from the stored samples.

3.5. Comparison of the PHASE Transition Temperatures

FIG. 2 shows comparative differential-calorimetric (DSC) measurements of the phase transition of the solid body-supported bilayer, consisting of the synthetic lipid dielaidoyl-sn-3-glycero-3-phosphocholine (termed DEPC below) on a nonoptimized solid-body surface (prepared in accordance with the prior art, e.g. C. Naumann, T. Brumm, T. M. Bayerl, Biophys. J., 1992, 63, 1314), or on a surface which has been optimized by means of the above step (as described in the example), as compared with the natural analog, i.e. the DEPC vesicles without any solid body support (prepared in accordance with the prior art by swelling the lipid and then extruding it, as described in M. J. Hope, M. B. Bally, G. Web, P. R. Cullis, Biochim. Biophys. Acta 1985, 812, 55).

These results show a marked shift and broadening of the phase transition in the case of the nonoptimized surface as compared with the (solid body-free) DEPC vesicles. The phase transition temperature is not affected on the optimized surface. The phase transition itself is only slightly broadened as compared with the natural analog.

FIG. 4 shows DSC measurements performed on DEPC lipid membranes which are located on different complex surfaces. In these experiments, all the examples depicted show virtually no shift in the phase transition temperature and only a slight broadening of the phase transition itself.

3.6. Comparative Deuterium (²H)-NMR Measurements

FIG. 3 shows deuterium (²H)-NMR measurements performed on chain-deuterated dipalmitoylphosphatidylcholine (DPPC)-d8 (7,7′,8,8′)-coated nonporous silicate particles with and without an optimized surface as compared with natural DPPC-d8 (7,7′,8,8′) vesicles. The quadrupole splitting of the signal originating from the selectively deuterated lipid chains is a measure of the molecular order in the bilayer (Seelig, J. Quarterly Reviews of Biophysics 1977, 10, 353-418). The unmodified surface gives rise to two splits, a phenomenon which has already been explained as being due to the asymmetry in the molecular order in the inner and outer monolayers of the bilayer which is caused by the immediate vicinity of the solid-body surface (M. Hetzer, S. Heinz, S. Grage, T. M. Bayerl, Langmuir, 1998, 14, 982-984). The twofold split is not seen either in the case of the optimized system or in the case of the natural analog (vesicle) and is therefore to be regarded as proof of an equivalent in the molecular order in both monolayers of the bilayer.

1. Method for immobilizing lipid layers, in particular double lipid layers, on surfaces of pulverulent solid bodies, characterized in that the solid-body surfaces are modified such that the properties, in particular with regard to diffusivity, of the lipid layers which have been deposited on them to a large extent correspond to those of lipid layers which have not been immobilized.

2. Method according to claim 1, comprising the following procedural steps:
a) modifying the solid-body surface with molecules in order to form a surface area which is essentially hydrophilic, and
b) depositing the lipid layers on the modified solid-body surface.

3. Method according to claim 2, characterized in that the modification of the solid-body surface comprises the following substeps:
aa) functionalizing the solid-body surface and/or
ab) adsorbing or chemisorbing interacting molecules.

4. Method according to claim 2 or claim 3, characterized in that the modification of the solid-body surface comprises the following substeps:
aa) functionalizing the solid-body surface and/or
ab) adsorbing or chemisorbing interacting molecules, and
ac) adsorbing or chemisorbing additional interacting molecules.

5. Method according to claim 3 or claim 4, characterized in that the solid-body surface is functionalized by applying aminofunctions, epoxyfunctions, haloalkylfunctions and/or thiofunctions, with the solid-body surface being treated, in particular, with aminofunctional, epoxyfunctional, haloalkylfunctional and/or thiofunctional molecules.

6. Method according to one of claims 3 to 5, characterized in that silanes are used for the functionalization.

7. Method according to one of claims 3 to 6, characterized in that mercaptans and/or disulfides, in particular alkyl disulfides, are used for the functionalization.

8. Method according to one of claims 3 to 7, characterized in that N-(2-aminoethyl)-3-aminopropyltrimethoxysilane (EDA), polyethylenimine (PEI) and/or cysteamine, in particular cysteamine hydrochloride, is/are used for the functionalization.

9. Method according to one of claims 3 to 8, characterized in that polymers, in particular polyelectrolytes, preferably anionic polyelectrolytes, polyampholytes, preferably proteins, and/or polyzwitterions are used as interacting molecules.

10. Method according to one of claims 3 to 9, characterized in that polystyrenesulfonate (PSS), in particular Na-polystyrenesulfonate, and/or poly(styrene-co-maleic anhydride) (PSPMA) is/are used as interacting molecule(s).

11. Method according to one of the preceding claims, characterized in that, in connection with modifying the solid-body surface, further molecules, in particular functional molecules, are inserted into the surface.

12. Method according to claim 11, characterized in that the functional molecules are dyes, in particular fluorescent dyes, and/or enzymically, chemically and/or photochemically reactive molecules.

13. Method according to one of the preceding claims, characterized in that the solid bodies are essentially porous pulverulent solid bodies.

14. Method according to one of the preceding claims, characterized in that the solid bodies are pulverulent silicates and/or pulverulent polymers.

15. Method according to one of the preceding claims, characterized in that the solid bodies are pulverulent magnetic particles and/or core-shell polymer particles.

16. Method according to one of the preceding claims, characterized in that the solid bodies have been applied to an essentially planar support or are applied to this support.

17. Method according to one of the preceding claims, characterized in that, in particular in connection with the modification of their surface, the solid bodies are used in the form of a dispersion, preferably in the form of a dispersion in water and/or alcohol.

18. Method according to one of the preceding claims, characterized in that the solid-body surface is a silicate surface, a semiconductor surface, in particular silicon surface, precious metal surface, in particular gold surface, and/or polymer surface, in particular polystyrene surface.

19. Method according to one of the preceding claims, characterized in that the lipid layers are double lipid layers, in particular lipid membranes.

20. Method according to one of the preceding claims, characterized in that the lipid layers are composed of lipids, lipid derivatives, lipid-like substances and/or lipid-analogous substances.

21. Method according to one of the preceding claims, characterized in that the lipid layers additionally contain peptides, proteins, nucleic acids, ionic or nonionic surfactants and/or polymers.

22. Method according to one of the preceding claims, characterized in that the lipid layers contain proteins, in particular enzymes, receptors and/or ligands, which are present superficially, such that they span the layer and/or such that they are embedded in it.

23. Method according to one of the preceding claims, characterized in that at least some of the lipid layers are composed of membrane fragments derived from natural cells.

24. Method according to one of the preceding claims, characterized in that the lipid layers are deposited on the modified solid-body surface by lipid vesicles being fused.

25. Modified solid-body surface which can be prepared by a method according to at least one of claims 1 to 18.

26. Lipid layer which is immobilized on a modified solid-body surface which can be prepared by a method according to at least one of claims 1 to 24.

27. Kit for preparing immobilized lipid layers on solid-body surfaces, at least comprising a modified solid-body surface according to claim 25 and/or reagents for preparing at least one modified solid-body surface according to claim 25, and also, preferably, reagents for depositing lipid layers on the modified solid-body surface according to at least one of claims 19 to 24.
